(19) United States
(12) Patent Application Publication
(10) Pub. No.: US 2021/0259580 A1
(43) Pub. Date: Aug. 26, 2021

(54) GAIT ANALYSIS METHOD, COMPUTER READABLE STORAGE MEDIUM AND ELECTRONIC DEVICE

(71) Applicant: SALTED CO., LTD., Seoul (KR)

(72) Inventors: Hyung Jin CHO, Seoul (KR); Kyung Hoon KANG, Seoul (KR)

(73) Assignee: SALTED CO., LTD., Seoul (KR)

(21) Appl. No.: 17/317,334

(22) Filed: May 11, 2021

Related U.S. Application Data
(63) Continuation of application No. PCT/KR2019/016295, filed on Nov. 25, 2019.

(30) Foreign Application Priority Data
Dec. 21, 2018 (KR) 10-2018-0167135
Feb. 28, 2019 (KR) 10-2019-0023629

(51) Int. Cl. A61B 5/11; A43B 3/00; A61B 5/00; G01L 5/00

(52) U.S. Cl. CPC A61B 5/112; G01L 5/0052; A61B 5/6807; A43B 3/0005

(57) ABSTRACT

A gait analysis method to be performed by an electronic device is disclosed. The method includes receiving a first sensing value of a first sensor unit and a second sensing value of a second sensor unit sensed for each time stamp from a shoe including the first sensor unit corresponding to a ball of a foot or a toe and the second sensor unit corresponding to a heel, determining a landing method of a user of the shoe based on the first sensing value and the second sensing value, calculating a ground contact starting point of a stance by a first method if the determined landing method is a first landing method, and calculating a ground contact starting point of a stance by a second method different from the first method if the determined landing method is a second landing method.

### CROSS-REFERENCE TO RELATED APPLICATION

This application is a Continuation of International Application No. PCT/KR2019/016295 filed on Nov. 25, 2019, which claims benefit of priority to Korean Patent Application No. 10-2018-0167135 filed on Dec. 21, 2018 and No. 10-2019-0023629 filed on Feb. 28, 2019, the entire content of which are incorporated herein by reference

### BACKGROUND

### 1. Field

The present invention relates to a gait analysis method, a computer readable storage medium, and an electronic device, and more particularly, it relates to a gait analysis method capable of accurately analyzing a user's gait using a smart shoe, a computer readable storage medium storing computer program for implementing the gait analysis method, and an electronic device, in which the gait analysis method is performed.

### 2. Description of the Related Art

The foot is an organ that supports all the weight of the human body and is an important organ that functions as a buffer to alleviate various impacts on the body. The human foot has 52 bones, which is about a quarter of the total bones, 64 muscles, 76 joints and 214 ligaments, which are intricately intertwined so that humans can stand upright for walking or exercising. Further, it is a very important organ, in which various nerves related to the functions of various internal organs of the human body are gathered on the sole of the human foot.

A shoe is a generic term for items worn on the feet, and can be used to protect and decorate the feet. Shoes are also worn in daily life, walking, running, and various exercises such as golf, and baseball.

### SUMMARY

On the other hand, recently, smart shoes, in which at least one sensor is installed in the shoes, are being developed. As the spread of smart shoes increases, it is necessary to develop a method for analyzing the gait of users using smart shoes.

The problem to be solved by the present invention is to provide a gait analysis method capable of accurately analyzing a user's gait using a smart shoe.

Another problem to be solved by the present invention is to provide a computer readable storage medium storing a computer program for implementing the gait analysis method.

Another problem to be solved by the present invention is to provide an electronic device, in which the gait analysis method is performed.

The problems of the present invention are not limited to the problems mentioned above, and other problems not mentioned will be clearly understood by those skilled in the art from the following description.

One aspect of the gait analysis method of the present invention for achieving the above object comprises performing by an electronic device, receiving a first sensing value of a first sensor unit and a second sensing value of a second sensor unit sensed for each time stamp from a shoe including the first sensor unit corresponding to a ball of a foot or a toe and the second sensor unit corresponding to a heel, determining a landing method of a user of the shoe based on the first sensing value and the second sensing value, calculating a ground contact starting point of a stance by a first method if the determined landing method is a first landing method, and calculating a ground contact starting point of a stance by a second method different from the first method if the determined landing method is a second landing method.

Wherein determining a landing method of a user of the shoe based on a first sensing value and the second sensing value comprises performing, if any one of the first sensing value in the first time stamp and the second sensing value in the first time stamp is greater than a first reference value, at least one of comparing the first sensing value in the first time stamp and the second sensing value in the first time stamp, and comparing the second sensing value in the first time stamp and a second reference value, and determining a landing method of a user of the shoe according to the comparison result.

Wherein determining a landing method of a user of the shoe according to the comparison result comprises determining that the user of the shoe uses Heel Strike if the second sensing value in the first time stamp is greater than the first sensing value in the first time stamp.

Wherein determining a landing method of a user of the shoe according to the comparison result comprises determining that the user of the shoe uses Midfoot Strike if the first sensing value in the first time stamp is greater than the second sensing value in the first time stamp, and the second sensing value in the first time stamp is greater than a second reference value.

Wherein determining a landing method of a user of the shoe according to the comparison result comprises determining that the user of the shoe uses Forefoot Strike if the first sensing value in the first time stamp is greater than the second sensing value in the first time stamp, and the second sensing value in a first time stamp and the second sensing value in a second time stamp consecutive to the first time stamp is less than a second reference value.

Wherein the first landing method is Heel Strike, a third time stamp is located immediately before the first time stamp, and the first method determines a first time between the first time stamp and the third time stamp as a ground contact starting point of a stance by considering the second sensing value in the first time stamp and the second sensing value in the third time stamp.

Wherein the second landing method is Midfoot Strike or Forefoot Strike, a third time stamp is located immediately before the first time stamp, the second method determines a second time between the first time stamp and the third time stamp as a ground contact starting point of a stance by considering the first sensing value in the first time stamp and the first sensing value in the third time stamp.

Wherein a fifth time stamp is located immediately after a fourth time stamp, and the gait analysis method further comprises calculating a ground contact starting point of a stance, and determining a third time between the fourth time stamp and the fifth time stamp as a ground contact ending point of a stance by considering the first sensing value in the fourth time stamp and the first sensing value in the fifth time stamp if the first sensing value in the fourth time stamp is greater than a third reference value and the first sensing value in the fifth time stamp is less than a third reference value,

Wherein the first sensor unit includes at least two sensors, and the first sensing value is a collection of individual sensing values sensed by each of the at least two sensors, the fifth time stamp is located immediately after the fourth time stamp, and the gait analysis method further comprises calculating a ground contact starting point of a stance, determining a third time between the fourth time stamp and the fifth time stamp as a ground contact ending point of a stance by considering correction values of individual sensing values in the fourth time stamp and correction values of individual sensing values in the fifth time stamp if a correction value generated using the individual sensing values in the fourth time stamp is greater than a fourth reference value, and a correction value generated using the individual sensing values in the fifth time stamp is less than a fourth reference value.

wherein the electronic device is a tread mill, and the gait analysis method further comprises receiving a track velocity of the tread mill, calculating a stride time between a ground contact ending point of a user's previous stance and a ground contact starting point of a user's current stance, and calculating a stride length of the user using the track velocity and the stride time.

Wherein the first landing method is Heel Strike, and the first method is to calculate a ground contact starting point of the stance based on a second sensing value, the second landing method is Forefoot Strike or Midfoot Strike, and the second method is to calculate a ground contact starting point of the stance based on the first sensing value.

One aspect of the computer readable storage medium of the present invention for achieving the above other object stores instructions of a computer program for implementing the gait analysis method described above.

One aspect of the electronic device of the present invention for achieving the above another object comprises a communication module, a processor, and a memory, wherein the memory stores instructions causing the communication module to receive a first sensing value of a first sensor unit and a second sensing value of a second sensor unit sensed for each time stamp from a shoe including the first sensor unit corresponding to a ball of a foot or a toe and the second sensor unit corresponding to a heel, wherein the memory stores instructions causing the processor to determine a landing method of a user of the shoe based on the first sensing value and the second sensing value, calculate a ground contact starting point of a stance by a first method if the determined landing method is a first landing method, and calculate a ground contact starting point of a stance by a second method different from the first method if the determined landing method is a second landing method.

wherein the electronic device is a tread mill, and the memory further stores instructions causing the processor to receive a track velocity of the tread mill, calculate a stride time between a ground contact ending point of a user's previous stance and a ground contact starting point of a user's current stance, and calculate a user's stride length using the track velocity and the stride time.

Details of other embodiments are included in the detailed description and drawings.

### BRIEF DESCRIPTION OF THE DRAWINGS

These and/or other aspects will become apparent and more readily appreciated from the following description of the embodiments, taken in conjunction with the accompanying drawings in which:

FIGS. 1A and 1B are side views for describing shoes according to some embodiments of the present invention;

FIG. 2 is a plan view for describing the outsole of FIG. 1A;

FIG. 3 is a cross-sectional view taken along line A-A of FIG. 1A;

FIG. 4 is an exploded perspective view of the sensing system of FIG. 3;

FIG. 5 illustrates the flexible circuit board of FIG. 3;

FIG. 6 is a cross-sectional view taken along B-B of FIG. 5;

FIG. 7 is a view for describing the support plate of FIG. 3;

FIG. 8 is a diagram illustrating a relationship between a shoe and an electronic device according to some embodiments of the present invention;

FIG. 9 is a diagram for describing a tread mill as an example of the electronic device of FIG. 8;

FIG. 10 is a flowchart for describing a gait analysis method according to some embodiments of the present invention;

FIG. 11 is a flowchart for specifically describing step S720 of determining a user's landing method based on the first sensing value and the second sensing value of FIG. 10;

FIG. 12 is exemplary data for describing a gait analysis method according to some embodiments of the present invention;

FIG. 13 is exemplary data for describing a gait analysis method according to some embodiments of the present invention; and

FIG. 14 is an exemplary user interface displayed on an electronic device according to some embodiments of the present invention.

### DETAILED DESCRIPTION

Hereinafter, preferred embodiments of the present invention will be described in detail with reference to the accompanying drawings. Advantages and features of the present invention, and methods for achieving them will be clarified with reference to embodiments described below in detail together with the accompanying drawings. However, the present invention is not limited to the embodiments disclosed below, but may be implemented in various different forms, and only the embodiments allow the publication of the present invention to be complete, and are provided to fully inform those skilled in the technical field to which the present invention pertains of the scope of the invention, and the invention is only defined by the scope of the claims. The same reference numerals refer to the same elements throughout the specification.

When elements are referred to as “on” or “above” of other elements, it includes not only when directly above of the other elements, but also other elements intervened in the middle. On the other hand, when elements are referred to as “directly on” or “directly above,” it indicates that no other element is intervened therebetween.

The spatially relative terms “below,” “beneath,” “lower,” “above,” “upper,” etc., as shown in figures, can be used to easily describe the correlation of components or elements with other components or elements. The spatially relative terms should be understood as terms including the different direction of the element in use or operation in addition to the direction shown in the figure. For example, if the element shown in the figure is turned over, an element described as “below” or “beneath” the other element may be placed “above” the other element. Accordingly, the exemplary term “below” can include both the directions of below and above. The element can also be oriented in other directions, so that spatially relative terms can be interpreted according to the orientation.

Although the first, second, etc. are used to describe various components, elements and/or sections, these components, elements and/or sections are not limited by these terms. These terms are only used to distinguish one component, element, or section from another component, element or section. Therefore, first component, the first element or first section mentioned below may be a second component, second element, or second section within the technical spirit of the present invention.

The terminology used herein is for describing the embodiments and is not intended to limit the present invention. In the present specification, the singular form also includes the plural form unless otherwise specified in the phrase. As used herein, “comprises” and/or “comprising” means that the elements, steps, operations and/or components mentioned above do not exclude the presence or additions of one or more other elements, steps, operations and/or components.

Unless otherwise defined, all terms (including technical and scientific terms) used in the present description may be used with meanings that can be commonly understood by those of ordinary skill in the art, to which the present invention belongs. In addition, terms defined in a commonly used dictionary are not interpreted ideally or excessively unless explicitly defined specifically.

Hereinafter, exemplary embodiments of the present invention will be described in detail with reference to the accompanying drawings, and in the description with reference to the accompanying drawings, the same or corresponding elements are assigned the same reference numbers regardless of reference numerals, and the description overlapped therewith will be omitted.

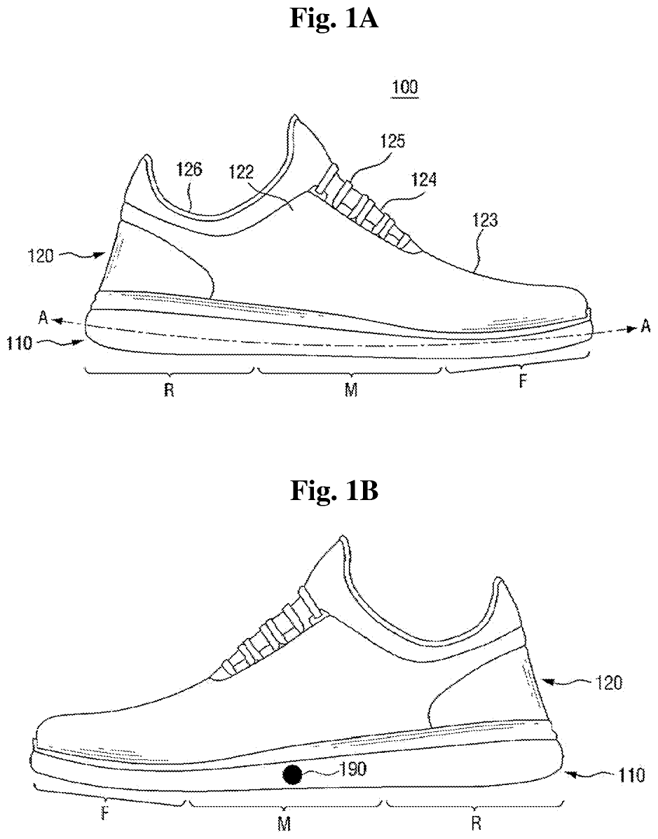

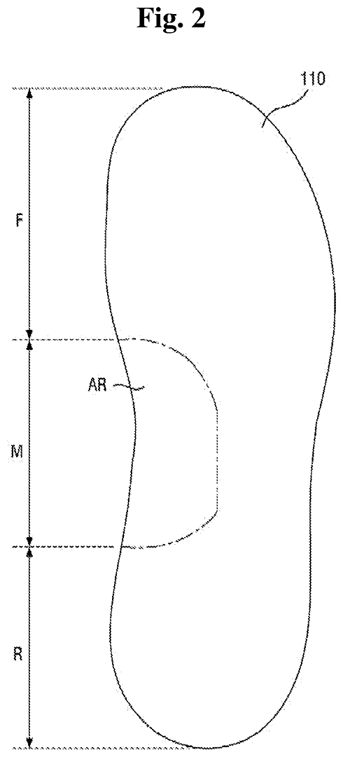

FIGS. 1A and 1B are side views for describing exemplary shoes used in a gait analysis method according to some embodiments of the present invention. That is, FIG. 1A is a side view viewed from the outside of the shoe, and FIG. 1B is a side view viewed from the inside of the shoe. FIG. 2 is a plan view for describing the outsole of FIG. 1A.

In FIGS. 1A and 1B, sports shoes are exemplarily described, but the present invention is not limited thereto. It may be applied to various types of sport shoes, for example, running shoes, tennis shoes, baseball shoes, volleyball shoes, soccer shoes, etc., and may also be applied to various types of shoes such as loafers, sneakers, straight tips, wing tips, and monk straps.

Referring to FIGS. 1A, 1B, and 2, the shoe 100 includes an outsole 110, an insole, an upper structure 120, and the like.

The outsole 110 is located in the lower portion of the shoe 100 and refers to a portion in contact with the ground. The outsole 110 may be made of a material, for example, such as leather, rubber, silicone, but is not limited thereto.

Further, the outsole 110 may include, for example, a forefoot area (F), a rear foot area (R), a mid foot area (M) arranged between the forefoot area (F) and the rear foot area (R). The ratio of the forefoot area (F), the mid foot area (M), and the rear foot area (R) may be, for example, F:M:R=40:30:30.

On the other hand, the arch area (AR) of the outsole 110 is a part corresponding to the arch area of the foot. The arch area (AR) may be a part of the mid foot area (M), and for example, may be arranged in the inner side of the mid foot area (M) (i.e., a direction, in which the other foot is located).

The upper structure 120 is connected and/or fixed to the outsole 110, and defines a space for the foot to enter. The upper structure 120 may be formed of one or more parts of, for example, leather, artificial leather, natural or synthetic fabric, polymer sheet, polymer foam material, mesh fabric, felt, non-quilted polymer, or rubber material, but is not limited thereto.

The upper structure 120 includes a side area 122, an instep area 123, and the like.

The side area 122 is arranged to extend along the side of the foot.

The instep area 123 is formed to correspond to the upper surface or the instep area of the foot. In addition, a space 124 having a lace 125 is formed in the instep area 123, and the overall dimensions of the shoe 100 can be modified using these. That is, a closing mechanism is applied so that the shoe 100 is well worn on the foot.

In addition, the foot enters the shoe 100 through the opening 126.

Meanwhile, an insole is arranged on the outsole 110. The insole is the surface that the foot directly contacts.

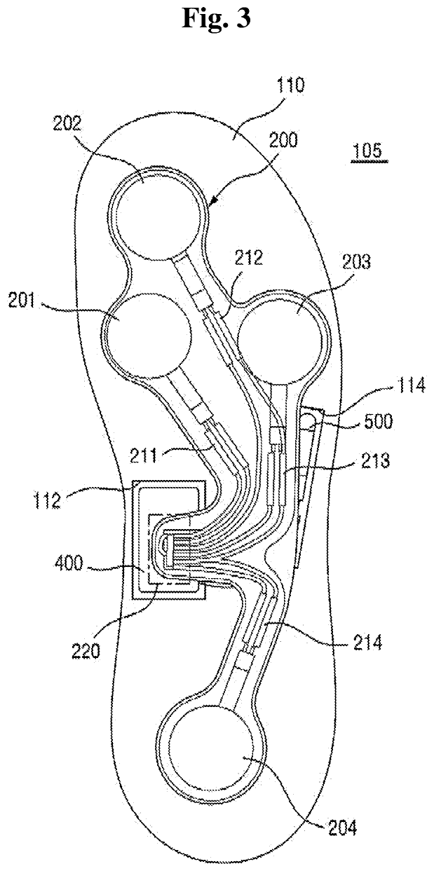

On the other hand, in the outsole 110 of the shoe according to some embodiments of the present invention, a completely embedded sensing system (see 105 in FIG. 3) is installed. The sensing system 105 may sense pressure generated by a foot using a plurality of sensors, and communicate with an external device (i.e., an electronic device) using an antenna. Since the sensing system 105 is completely embedded in the outsole 110, the outsole 110 is detachable from the upper structure 120 for manufacturing and post-sale management. The detachable method may be changed according to the bonding method (mechanical bonding, chemical bonding, etc.) of the outsole 110 and the upper structure 120.

Further, as illustrated in FIG. 1B, a state indicator 190 for displaying the state of the shoe may be installed on the outsole 110 of the shoe. For example, the state indicator 190 may be anything that can display the state of the shoe. For example, the state indicator 190 may inform the user of the state of the shoe by using at least one of visual, auditory, and tactile senses. The state indicator 190 may be a visual-related product such as LED or OLED. The state indicator 190 may change the color of the LED according to the state of the shoe. For example, the state indicator 190 may indicate blue in the connection standby mode (or unconnected state), the state indicator 190 may indicate red in the confirmation mode (i.e., ready state for pairing), and the state indicator 190 may indicate green in the pairing mode (i.e., state, in which pairing is completed).

Further, the state indicator 190 may be installed inside the outsole 110 as shown in FIG. 1B, but is not limited thereto. For example, it may be arranged on the bottom of the outsole 110 (i.e., a surface facing the ground), or may be installed outside the outsole 110.

Further, although it is illustrated as being installed in the mid foot area (M) of the outsole 110, it is not limited thereto. For example, it may be installed in the forefoot area (F) or the rear foot area (R) of the outsole 110.

Meanwhile, when the state indicator 190 is a tactile device, when the user presses a button to check the state of the shoe, the vibration pattern (or vibration interval) may vary according to the state of the shoe.

Meanwhile, when the state indicator 190 is an auditory device, when the user presses a button to check the state of the shoe, a guide comment, sound, or music, etc. that varies according to the state of the shoe may be output.

Next, the sensing system 105 will be described in detail with reference to FIGS. 3 to 7.

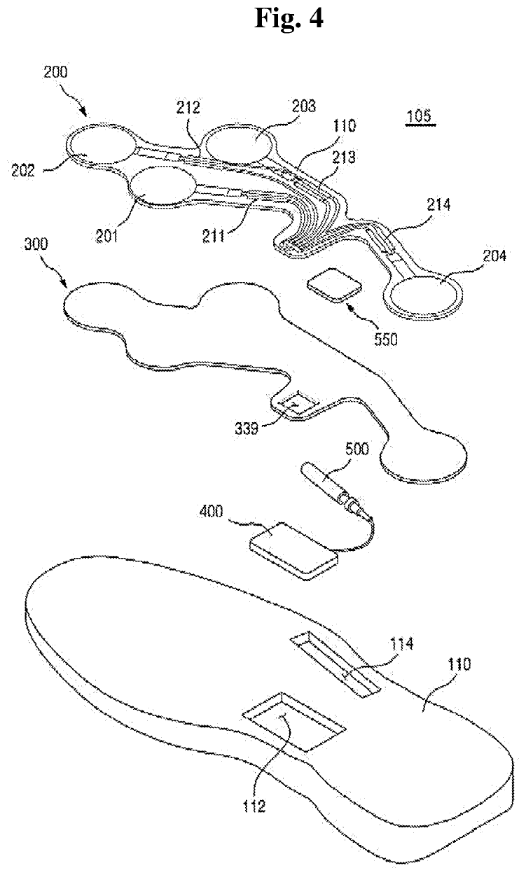

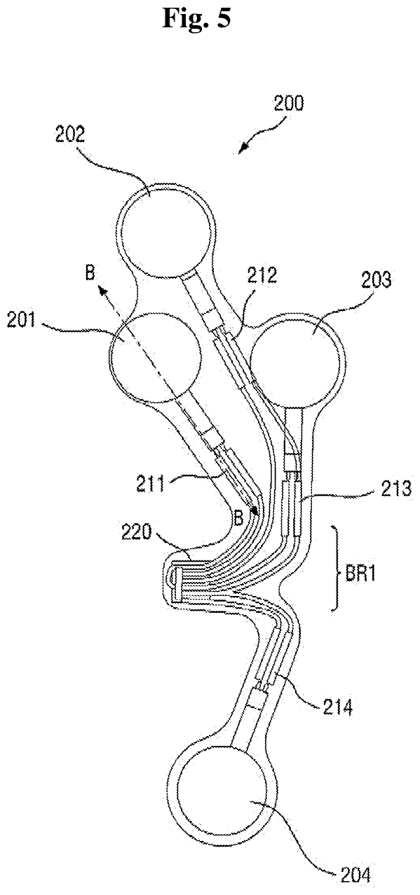

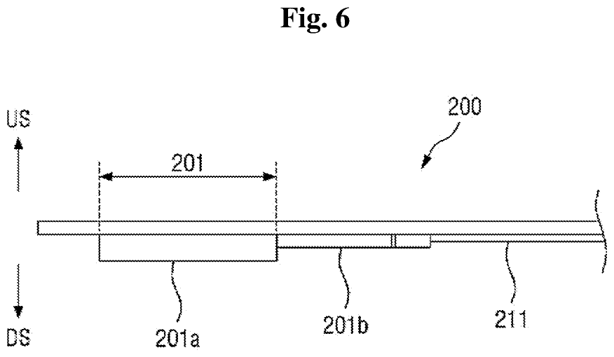

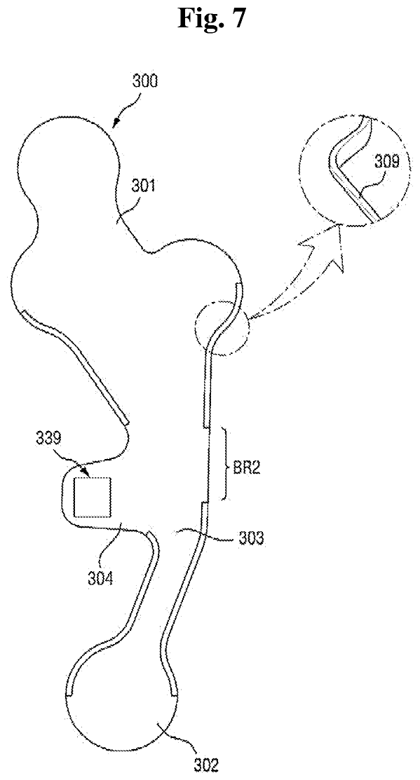

FIG. 3 is a cross-sectional view taken along line A-A of FIG. 1A. FIG. 4 is an exploded perspective view of the sensing system of FIG. 3. FIG. 5 illustrates the flexible circuit board of FIG. 3. FIG. 6 is a cross-sectional view taken along B-B of FIG. 5. FIG. 7 is a view for describing the support plate of FIG. 3.

First, referring to FIGS. 3 to 5, the sensing system 105 may include a flexible circuit board 200, a binder 550, a support plate 300, a control module 400, an antenna 500, and the like.

The flexible circuit board 200 includes a plurality of sensing areas 201, 202, 203, and 204, in which a plurality of sensors may be installed, and wirings 211, 212, 213, and 214 connected to the plurality of sensors.

Among the sensing areas 201, 202, 203, and 204, the first sensing area 201 and the third sensing area 203 may correspond to the ball of the foot, the second sensing area 202 may correspond to the big toe of the foot, and the fourth sensing area 204 may correspond to the heel. Here, the positions and numbers of the plurality of sensing areas 201, 202, 203, and 204 may vary according to design. For example, the number of sensing areas 201, 202, 203, and 204 may be 5 or more or 3 or less. Further, the sensing areas 201, 202, 203, and 204 may be arranged at a position corresponding to the second toe or the third toe instead of the big toe, or may correspond to a position corresponding to the mid foot area. Hereinafter, it will be described that one sensor 201a, 202a, 203a, 204a is arranged in each of the sensing areas 201, 202, 203, and 204, but is not limited thereto. That is, not one sensor may be arranged in each sensing area 201, 202, 203, 204, but two or more sensors may be arranged. Further, in the shoes according to some embodiments of the present invention, the sensor may be a film-type pressure sensor. Depending on the design, other types of sensors may be arranged.

The plurality of sensors may be arranged closer to the top surface (opposite of the bottom surface) of the outsole 110 than the bottom surface of the outsole 110. This is to make it easier to recognize the pressure from the foot.

The wirings 211, 212, 213, and 214 may start in the common area 220 and branch in the direction of each of the sensing areas 201, 202, 203, and 204.

For example, the wirings 211, 212, 213, and 214 may have an inverted C shape or a right parenthesis (i.e., “)” shape) as shown. That is, the wirings 211, 212, 213, and 214 may be formed to start in the common area 220 and bend in the outer direction of the shoe to reach each of the sensing areas 201, 202, 203, and 204. By having such a shape, it is possible to stably collect each of the wirings 211, 212, 213, 214 in the common area 220 and prevent disconnection of the wirings 211, 212, 213, and 214.

Here, the wirings 211, 212, 213, and 214 may be electrically connected to the control module 400 through the common area 220.

Although described later, the common area 220 may be formed inside the arch area (AR) of the shoe.

Further, as illustrated in FIG. 6, the sensor 201a may be arranged in the downward direction (DS) of the sensing area 201. For example, the wiring 201b from the sensor 201a may be directly connected to the wiring 211 in the wiring area. These wirings 201b and 211 may also be arranged in the downward direction (DS). Here, the upward direction (US) is the direction, in which the user of the shoe 100 is located, and the downward direction (DS) is the opposite of the upward direction (US) and is the ground direction. In this way, since the wirings 201b and 211 and the sensor 201a are directed downward direction (DS) and facing the support plate 300, durability may be improved. Since the sensing system 105 is formed to be completely embedded in the outsole 110, for example, when the wirings 201b and 211 are directed upward direction (US), the wirings 201b and 211 are directly connected to the outsole 110. In this case, the wires 201b and 211 cause friction with the outsole 110, and thus may be easily cut off. On the other hand, when the wirings 201b and 211 face the support plate 300, the possibility of such a disconnection is lowered.

Referring back to FIGS. 3, 4, and 7, the support plate 300 is arranged in the downward direction of the flexible circuit board 200. In FIG. 7, the support plate 300 is illustrated to have a shape similar to that of the flexible circuit board 200, but is not limited thereto. The support plate 300 connects the support units 301 and 302 arranged in the lower portion of the sensing areas 201, 202, 203, and 204 of the flexible circuit board 200 and the support units 301 and 302 to each other, and includes a connection unit 303 arranged in the lower portion of the wirings 211, 212, 213, and 214 of the flexible circuit board 200. Further, the flexible circuit board 200 may further include a branch unit 304 formed to extend from the connection unit 303 in a direction, in which the control module 400 is installed. However, the shape of the support plate 300 may be any shape as long as it can perform the functions mentioned below.

First, the support plate 300 serves to increase the sensing sensitivity of a sensor (e.g., 201a) installed on the flexible circuit board 200.

When the user walks, runs, or exercises, the user's feet step on the sensor 201a (film-type pressure sensor). However, since the sensor 201a is embedded in the outsole 110, if there is no support plate 300, the sensor 201a directly presses the outsole 110 when the user's feet step on the sensor 201a. However, when the outsole 110 is formed of a soft material (e.g., rubber, silicone) that can prevent impact, the sensor 201a meets the soft material. Accordingly, the sensing sensitivity of the sensor 201a is deteriorated. Accordingly, a support plate 300 made of a material having a strength higher than that of the outsole 110 is installed under the sensing areas 201, 202, 203, 204 of the flexible circuit board 200. Accordingly, when the user's foot presses the sensor 201a, the sensor 201a directly contacts the support units 301 and 302 of the support plate 300, which is not a soft material. Accordingly, it is possible to increase the sensing sensitivity of the sensor 201a.

Further, the support plate 300 may have a strength higher than that of the flexible circuit board 200.

In addition, the support plate 300 may hold the position of the flexible circuit board 200.

Since the flexible circuit board 200 is a soft material that can bend and the sensing system 105 is embedded in the outsole 110, the flexible circuit board 200 cannot be placed in the proper position within the outsole 110. Further, it is not easy to know from the outside whether or not it is placed in the proper position.

However, the support plate 300 includes a protrusion unit 309 so that the flexible circuit board 200 can be easily positioned.

The protrusion unit 309 may extend in an upward direction (i.e., a direction, in which the flexible circuit board is located). The flexible circuit board 200 may be fixed in position by the protrusion unit 309.

The protrusion unit 309 may be formed along the contour of the support plate 300, but is not limited thereto. It may be formed on the entire contour of the support plate 300 or may be formed only on a part of the contour. For example, as shown in FIG. 7, the protrusion unit 309 may be formed at a portion where the support unit 301 and the connection unit 303 are connected. The support unit 301 or 302 may be relatively wide along the shape of the flexible circuit board 200, and the connection unit 303 may be relatively narrow in consideration of an area, in which the control module 400 is to be formed. That is, the width of the support unit 301 is wider than the width of the connection unit 303. Alternatively, the width of the support unit 302 is wider than the width of the connection unit 303. Therefore, as shown in FIG. 7, since the protrusion unit 309 is formed at a portion where the support unit 301 and the connection unit 303 are connected to each other, or a portion where the support unit 302 and the connection unit 303 are connected to each other, even though the protrusion unit 309 is formed in a non-wide area, it may be easy to fix the position of the flexible circuit board 200.

Further, the branch unit 304 is formed to extend from the connection unit 303 of the support plate 300 in the direction, in which the control module 400 is installed. The branch unit 304 is arranged on the control module 400 to prevent damage to the control module 400 from unexpected external strong impact.

Meanwhile, a through hole 339 may be formed in the branch unit 304 of the support plate 300. The control module 400 and the flexible circuit board 200 may be electrically connected to each other through the through hole 339. That is, the common area 220, in which the wirings 211, 212, 213, and 214 of the flexible circuit board 200 are gathered, may be arranged to correspond to the through hole 339.

Meanwhile, depending on the design, the branch unit 304 of the flexible circuit board 200 may be omitted. In this case, there may be no other components between the flexible circuit board 200 and the control module 400, and thus, the flexible circuit board 200 and the control module 400 can be easily electrically connected to each other.

Further, the binder 550 serves to connect the flexible circuit board 200 and the support plate 300 to each other. As the binder 550, for example, various types of adhesives may be used. For example, solvent adhesives, pressure sensitive adhesives, heat sensitive adhesives, reactive adhesives, etc. may be used, but the present invention is not limited thereto.

Referring back to FIG. 4, the binder 550 may be formed, for example, on at least a part of the connection unit 303 of the support plate 300. That is, the binder 550 does not fix the entire flexible circuit board 200 and the support plate 300.

Specifically, the gait phase of the user can be largely divided into a stance phase and a swing phase. Here, the stance phase refers to a period, in which the foot is in contact with the ground, and the swing phase refers to a period, in which the foot is away from the ground. Again, the stance step may proceed in the order of an initial contact, a loading response, a mid stance, and a terminal stance. In addition, the swing step may be performed in the order of pre-swing and swing.

The initial contact and loading response require weight acceptance, the mid stance and terminal stance require single limb support, and the free swing and swing require stretching feet forward to move the weight forward (swing limb advancement).

Meanwhile, in the terminal stance step, the sensors arranged in the first and third sensing areas 201 and 203 (i.e., the sensors arranged in a position corresponding to the ball of the foot) may be pressed, and then, in the free swing step, only a sensor arranged in the second sensing area 202 (i.e., a sensor arranged in a position corresponding to the big toe) may be pressed. During the user's gait, the first and second sensing areas 201 and 203 may be bent in a process of being connected to the free swing from the terminal stance.

Here, referring to FIGS. 4 and 7, the binder 550 may be formed in at least a part of the connection unit 303 of the support plate 300. That is, the binder 550 may be formed between the first and second sensing areas 201 and 203 and the fourth sensing area 204. Alternatively, the binder 550 may not be formed in the sensing areas 201, 203, and 204, but may be formed only in at least a part of the connection unit 303. Alternatively, the binder 550 may be formed in a part of the connection unit 303 close to the branch unit 304 (see BR2 in FIG. 7). Alternatively, it may be formed in an area between the adjacent protrusion unit 309 among the connection unit 303 (see BR2 in FIG. 7).

Since the support plate 300 and the flexible circuit board 200 are connected (attached) in this way, in the process of being connected to the free swing from the terminal stance, even if the flexible circuit board 200 is bent, the support plate 300 is only slightly bent. Therefore, in this process, the flexible circuit board 200 and the support plate 300 are slightly separated. That is, the flexible circuit board 200 may be slightly lifted.

Accordingly, even if the support plate 300 has a higher strength than the flexible circuit board 200, durability of the flexible circuit board 200 and the support plate 300 can be secured in the user's gait step.

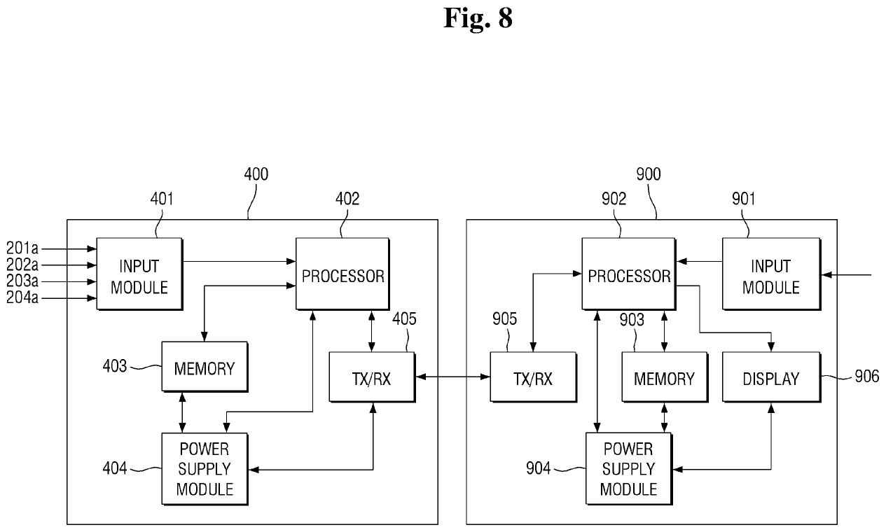

FIG. 8 is a diagram illustrating a relationship between a shoe and an electronic device according to some embodiments of the present invention. The configuration of the shoe (control module 400) shown in FIG. 8 and the configuration of the electronic device are exemplary, and are not limited thereto.

Referring to FIG. 8, the control module 400 may include an input module 401, a processor 402, a memory 403, a power supply module 404, a transmission/reception module 405, and the like. Each module inside may be individually housed (i.e., in the form of a single chip), or several may be housed in one (i.e., in the form of a composite chip).

The input module 401 receives a plurality of sensing signals provided from the plurality of sensors 201a to 204a. As described above, the plurality of sensors 201a to 204a may be film-type pressure sensors.

The processor 402 processes a plurality of input sensing signals. For example, the processor 402 may convert a data format suitable for storage in the memory 403 or may match a measurement time and a sensing signal. The processor 402 controls the memory 403, the power supply module 404, and the transmission/reception module 405.

The memory 403 may store a plurality of sensing signals according to time or may store signals processed by the processor 402.

The power supply module 404 may provide power to the processor 402, the memory 403, the transmission/reception module 405, and the like. Various types of primary and secondary batteries may be used as the power supply module 404. Lead, nickel-cadmium, nickel-hydrogen, lithium ion, lithium polymer batteries, etc. may be used as the secondary battery, but are not limited thereto. Alternatively, the power supply module 404 may include a module that generates power using a piezoelectric sensor (e.g., a piezo sensor) or the like.

Unlike shown, an additional sensor (not shown) may be installed in the shoe 100. For example, additional sensors may sense pedometer velocity and/or distance information, other velocity and/or distance data sensor information, temperature, altitude, atmospheric pressure, humidity, GPS data, accelerometer output or data, heart rate, pulse, blood pressure, body temperature, EKG data, EEG data, angular orientation (gyroscope-based sensor, etc.) and data related to angular orientation change. Alternatively, an additional sensor may sense data or information about a wide variety of other types of parameters, such as physical or physiological data related to the use of the shoe product or the user.

The control module 400 may communicate sensing signals or processed data with the electronic device 900 through the transmission/reception module 405.

The electronic device 900 may be a computing device for performing a gait analysis method according to some embodiments of the present invention, and is not limited to a type. For example, the electronic device may include, for example, at least one of a smartphone, a tablet PC, a mobile phone, a video phone, an e-book reader, a desktop PC, a laptop PC, a netbook computer, a workstation, a server, a PDA, a portable multimedia player (PMP), a MP3 player, a medical device, a camera, or a wearable device. Wearable devices include at least one of accessory types (e.g. watches, rings, bracelets, anklets, necklaces, glasses, contact lenses, or head-mounted-devices (HMD)), fabric or clothing integrals (e.g. electronic clothing), a body-attached type (e.g., a skin pad or a tattoo), or a bio-implantable circuit. In some embodiments, the electronic device may include, for example, at least one of a television, a digital video disk (DVD) player, audio, refrigerator, air conditioner, vacuum cleaner, oven, microwave, washing machine, air purifier, set-top box, home automation control panel, security control panel, media box (e.g. Samsung HomeSync™, Apple TV™, or Google TV™), game console (e.g., Xbox™, PlayStation™), an electronic dictionary, an electronic key, a camcorder, or an electronic frame.

In another embodiment, the electronic device may be a variety of exercise equipment (e.g., tread mill, stepper, bike, twister, etc.).

In another embodiment, the electronic device may include at least one of various medical devices (e.g., various portable medical measuring devices, such as blood glucose meter, heart rate meter, blood pressure meter, or body temperature meter, etc.), magnetic resonance angiography (MRA), magnetic resonance imaging (MRI), computed tomography (CT), camera, ultrasound device, navigation device, global navigation satellite system (GNSS), event data recorder (EDR), flight data recorder (FDR), automobile infotainment device, ship electronics equipment (e.g. marine navigation devices, gyro compasses, etc.), avionics, security devices, vehicle head units, industrial or home robots, drones, ATMs in financial institutions, POS (point of sales) in stores, or Internet of Things devices (e.g., light bulbs, various sensors, sprinkler devices, fire alarms, temperature controllers, street lights, toasters, exercise equipment, hot water tanks, heaters, boilers, etc.). According to some embodiments, the electronic device may include at least one of a piece of furniture, a building/structure or a vehicle, an electronic board, an electronic signature receiving device, a projector, or various measuring devices (e.g., water, electricity, gas, or a radio wave measuring device, etc.). In various embodiments, the electronic device may be flexible or may be a combination of two or more of the aforementioned various devices. The electronic device according to the embodiment of the present description is not limited to the above-described devices. In this description, the term user may refer to a person using an electronic device or a device using an electronic device (e.g., an artificial intelligence electronic device).

The electronic device 900 may include an input module 901, a processor 902, a memory 903, a power supply module 904, a communication module 905, a display 906, and the like.

The input module 901 may receive instructions/data and the like from a user.

The communication module 905 may receive a sensing signal or processed data from the shoe 100. Further, signals/data may be provided from components other than the shoe 100.

The processor 902 processes signals/data provided from the transmission/reception module 901. For example, according to time, a sensing signal and a video signal (e.g., a video signal obtained by measuring an exercise (motion) performed by wearing a shoe with a camera) may be matched with each other. Further, the processor 402 controls the memory 903, the power supply module 904, the communication module 905, the display 906, and the like.

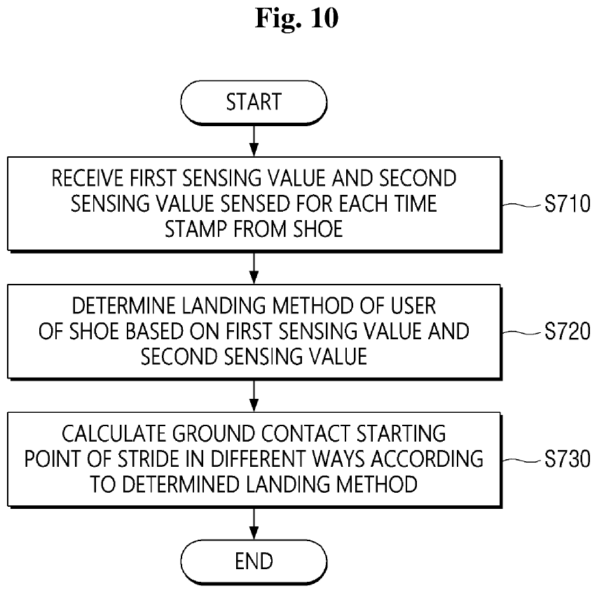

The memory 903 may store signals/data provided by the processor 902. Further, the memory 903 may include instructions of a computer program for implementing a gait analysis method to be described later using FIGS. 10 to 14.

For example, the memory stores instructions for the communication module 905 to receive a first sensing value of a first sensor unit and a second sensing value of a second sensor unit sensed for each time stamp from a shoe including a first sensor unit corresponding to a ball of a foot or a toe and a second sensor unit corresponding to a heel, and stores instructions for the processor to determines a landing method of the user of the shoe based on the first sensing value and the second sensing value, and calculate the ground contact starting point of the stance by a first method (for example, calculated based on the second sensing value) if the determined landing method is the first landing method (for example, Heel Strike), or calculate the ground contact starting point of the stance by a second method (for example, calculated based on the first sensing value) different from the first method if the determined landing method is the second landing method (for example, Forefoot Strike or Midfoot Strike).

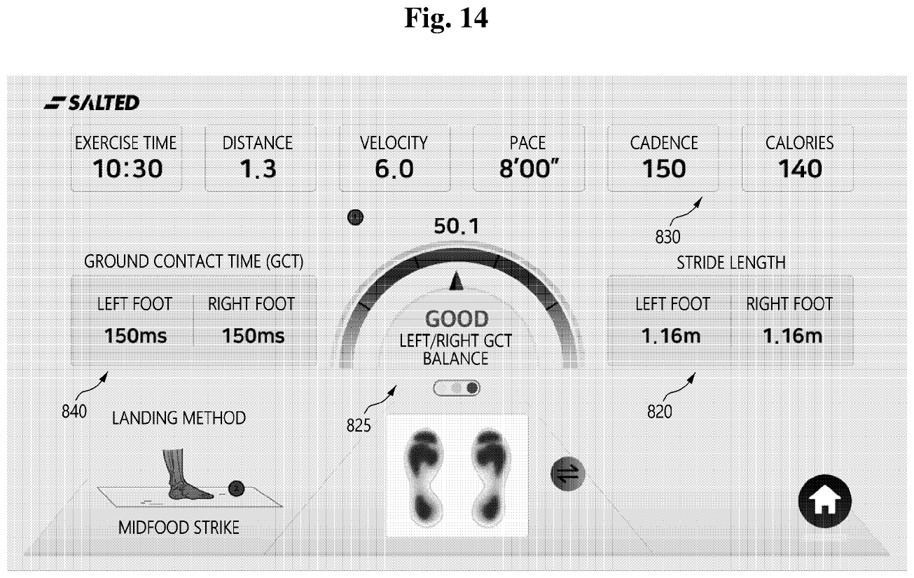

The power supply module 904 supplies power to the processor 902, the memory 903, the display 906, and the like. The display 906 shows signals/data generated by the processor 902 to the outside. An exemplary user interface displayed on display 906 is disclosed in FIG. 14.

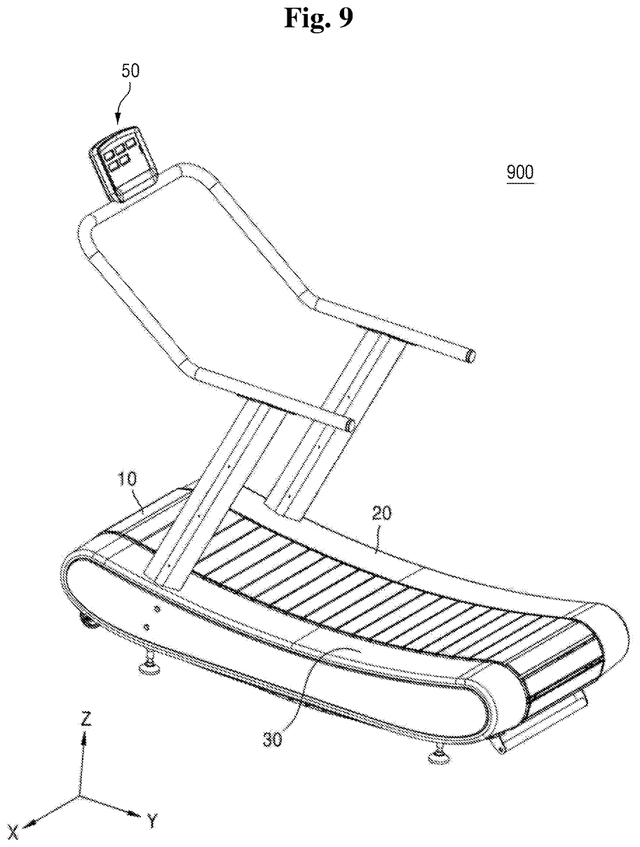

FIG. 9 is a diagram for describing a tread mill, which is an example of an electronic device according to some embodiments of the present invention.

Referring to FIG. 9, the tread mill includes a plurality of slats 10, first and second side covers 20 and 30, a management module 50, and the like. The slats 10 extend in a first direction (e.g., the X direction in FIG. 9) and may be arranged in a second direction perpendicular to the first direction (e.g., the Y direction in FIG. 9). When the user gaits (for example, walking or jogging) on the tread mill 1, the slat 10 may move along the second direction by the user's foot motion. The first and second side covers 20 and 30 are provided on both sides of the slat 10 in the longitudinal direction (first direction). The first and second side covers 20 and 30 may be provided to cover the first and second side frames. Since the tread mill is a device for exercising by using a belt rotating in an endless track, the shape of the slat 10, to which the user's foot contacts, is not limited to that shown in FIG. 9. That is, the shape of the slat 10 may be any shape as long as the user can safely move.

The management module 50 may include various configurations shown in FIG. 9 (for example, an input module 901, a processor 902, a memory 903, a communication module 905, a display 906, etc.). The management module 50 may control the operation of the tread mill (e.g., velocity control, angle control, etc.), receive an instruction (e.g., target velocity instruction, target angle instruction, program instruction, etc.) from a user, or provide various gait analysis data to the user.

FIG. 10 is a flowchart illustrating a gait analysis method according to some embodiments of the present invention. The gait analysis method may be performed by an electronic device.

Referring to FIG. 10, a first sensing value of a first sensor unit and a second sensing value of a second sensor unit sensed for each time stamp are received from a shoe 100 including a first sensor unit and a second sensor unit (S710).

Specifically, the first sensor unit may include at least one sensor corresponding to the ball of the foot or the toe, and the second sensor unit may include at least one sensor corresponding to the heel.

In the case of the shoe 100 described with reference to FIGS. 1a to 8, sensors 201a, 202a, 203a and 204a may be arranged in each of the plurality of sensing areas 201, 202, 203, and 204. In this case, the first sensor unit may include sensors (i.e., 201a, 202a, 203a) arranged in the sensing areas 201, 202, and 203 corresponding to the ball of the foot or the toe, and the second sensor unit may include a sensor 204a arranged in a sensing area 204 corresponding to the heel.

The shoe 100 and the electronic device 900 may communicate using short-range wireless communication (e.g., Bluetooth communication). Due to the limitation of the Bluetooth communication specification and the limitation of the power supply module (see 404 in FIG. 8) (i.e., using the primary or secondary battery), the first sensing value (S1(t)) and the second sensing value (S2(t)) can be sent per each time stamp. If the time stamp is generated, for example, in units of 33 ms, the first sensing value (S1(t)) and the second sensing value (S2(t)) sensed at Oms, 33 ms, 66 ms, 99 ms . . . are transmitted.

For convenience of description, S1(t) denotes the first sensing value sensed by the first sensor unit in the first time stamp (t), and S2(t) denotes the second sensing value sensed by the second sensor unit in the first time stamp (t). Similarly, S1(t+1) denotes a first sensing value sensed by the first sensor unit in a second time stamp (t+1) positioned immediately after the first time stamp (t), and S1(t−1) denotes a first sensing value sensed by the first sensor unit in a third time stamp (t−1) positioned immediately before the first time stamp (t). For example, when a time stamp is generated in units of 33 ms, if the first time stamp (t) is generated in 66 ms, the second time stamp (t+1) is generated in 99 ms, and the third time stamp (t−1) is generated in 33 ms.

Subsequently, based on the first sensing value and the second sensing value, the landing method of the user of the shoe is determined (S720).

Here, the landing method may include, for example, Heel Strike, Midfoot Strike, and Forefoot Strike, but are not limited thereto. Heel Strike refers to that the heel lands first when gaiting, Midfoot Strike refers to that the middle of the sole lands first when gaiting, and Forefoot Strike refers to that the fore foot lands first when gaiting.

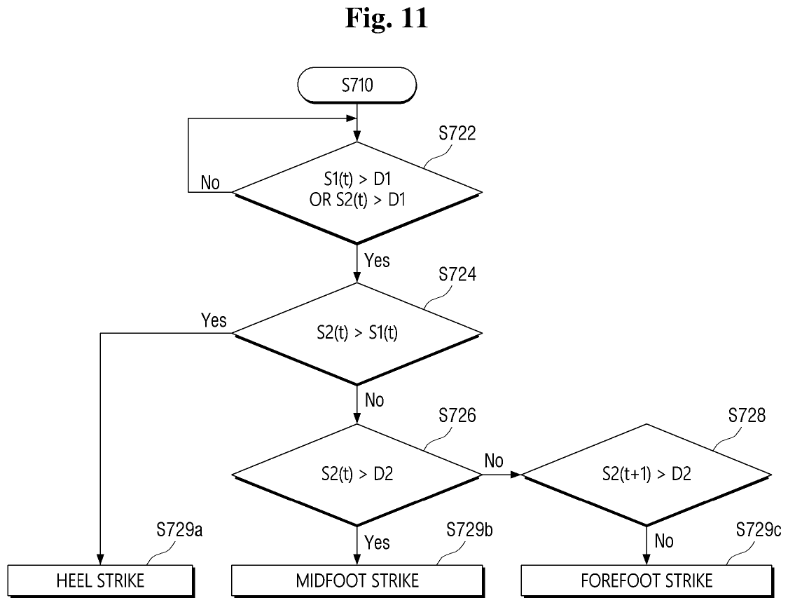

A method of determining the landing method will be described later in detail with reference to FIGS. 11 to 13.

Subsequently, the ground contact starting point of the stance is calculated in different ways according to the determined landing method (S730). That is, if the determined landing method is the first landing method, the ground contact starting point of the stance is calculated by the first method, and if the determined landing method is the second landing method, the ground contact starting point of the stance is calculated by a second method different from the first method.

The gait data used to analyze gait is diverse, and may include, for example, Cadence (i.e., steps per minute, revolutions per minute, gait velocity), ground contact time (GCT), vertical oscillation, gait time, gait velocity, gait length, gait period, and the like.

In particular, since the ground contact time (GCT) can be obtained by subtracting the ground contact starting point from the ground contact ending point, in order to accurately calculate the ground contact time (GCT), it is necessary to accurately predict the ground contact starting point and the ground contact ending point.

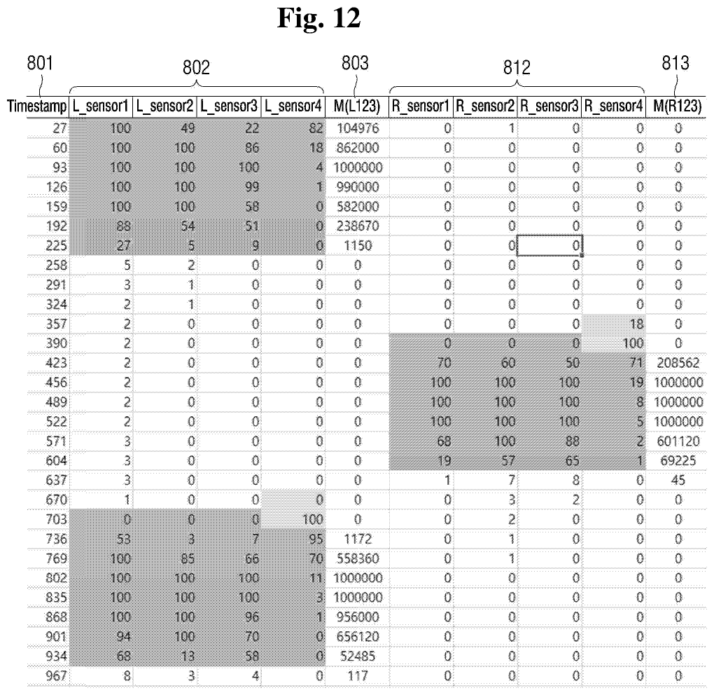

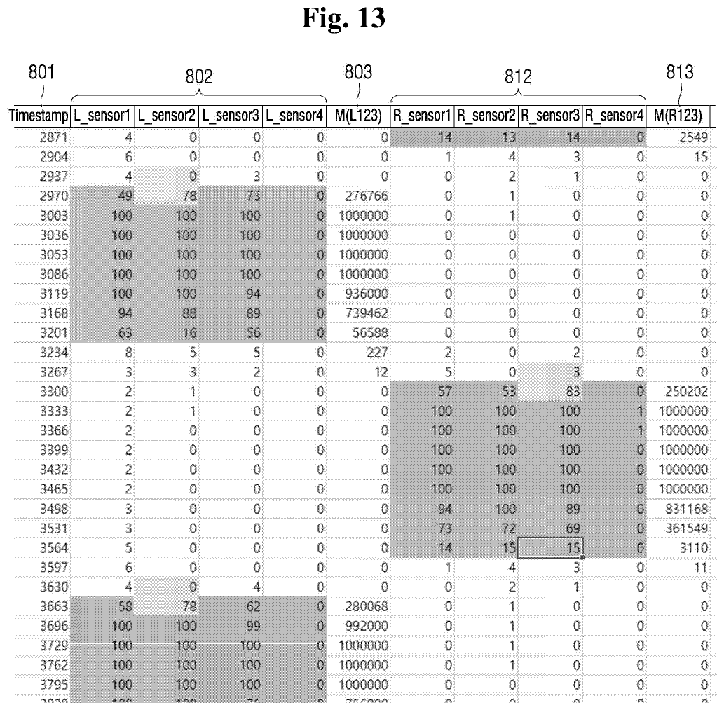

According to the gait analysis method according to some embodiments of the present invention, a ground contact starting point and a ground contact ending point can be predicted fairly accurately using sensing values provided by smart shoes equipped with a plurality of sensors. A method of predicting a ground contact starting point and a ground contact ending point will be described in detail later with reference to FIGS. 12 and 13.

FIG. 11 is a flowchart specifically illustrating a step of determining a user's landing method based on the first sensing value and the second sensing value (S720) of FIG. 10.

Referring to FIG. 11, it is checked whether any one of a first sensing value (S1(t)) in a first time stamp (t) and a second sensing value (S2(t)) in a first time stamp (t) is greater than the preset reference value (D1) (S722).

If it is less (NO), repeat step S722. That is, it is checked whether any one of the first sensing value (S1(t+1)) in the second time stamp (t+1) immediately after the first time stamp (t) and the second sensing value (S2(t+1)) in the second time stamp (t+1) is greater than the reference value (D1).

If it is greater (YES), the first sensing value (S1(t)) in the first time stamp (t) and the second sensing value (S2(t)) in the first time stamp (t) are compared (S724).

If the second sensing value (S2(t)) in the first time stamp (t) is greater than the first sensing value (S1(t)) in the first time stamp (t) (YES), it is determined that the user of the shoe uses Heel Strike (S729a).

If the second sensing value (S2(t)) in the first time stamp (t) is less than the first sensing value (S1(t)) in the first time stamp (t) (NO), the second sensing value S2(t) in the first time stamp (t) and the preset reference value (D2) are compared (S726).

If the second sensing value (S2(t)) in the first time stamp (t) is greater than the reference value (D2) (YES), it is determined that the user of the shoe uses Midfoot Strike (S729b).

If the second sensing value (S2(t)) in the first time stamp (t) is less than the reference value (D2) (NO), the second sensing value (S2(t+1)) in the second time stamp (t+1) and the reference value (D2) are compared (S728).

If the second sensing value (S2(t+1)) in the second time stamp (t+1) is less than the reference value (D2) (NO), it is determined that the user of the shoe uses Forefoot Strike (S729c).

If the second sensing value (S2(t+1)) in the second time stamp (t+1) is greater than the reference value (D2) (YES), the determination of the landing method is suspended.

In summary, any one of the first sensing value (S1(t)) in the first time stamp (t) and the second sensing value (S2(t)) in the first time stamp (t) is greater than the reference value (D1), at least one of comparing the first sensing value (S1(t)) in the first time stamp (t) and the second sensing value S2(t) in the first time stamp (t) with each other, and comparing the second sensing value (S2(t)) in the first time stamp (t) and the reference value (D2) is performed. Additionally, comparing the second sensing value (S2(t+1)) in the second time stamp (t+1) with the reference value (D2) may be further performed. According to the comparison result, the landing method of the user of the shoe is determined.

Further, when the user uses Heel Strike, the heel (corresponding to the second sensor unit) lands more strongly than the ball of the foot or the toe (corresponding to the first sensor unit) when the foot is landed. Therefore, when it is determined that the user uses Heel Strike, it is the case, in which the second sensing value (S2(t)) in the first time stamp (t) is greater than the first sensing value (S1(t)) in the first time stamp (t).

When the user uses Midfoot Strike or Forefoot Strike, the ball of the foot or toe (corresponding to the first sensor unit) lands more strongly than the heel (corresponding to the second sensor unit) when the foot is landed. Even in such a case, if the user uses Midfoot Strike, the heel weakly lands, but if the user uses Forefoot Strike, the heel hardly lands.

Therefore, when it is determined that the user uses Midfoot Strike, it is a case, in which the first sensing value (S1(t)) in the first time stamp (t) is greater than the second sensing value (S2(t)) in the first time stamp (t), and the second sensing value (S2(t)) in the first time stamp (t) is greater than the reference value (D2).

Further, when it is determined that the user uses Forefoot Strike, the first sensing value (S1(t)) in the first time stamp (t) is greater than the second sensing value (S2) in the first time stamp (t), and the second sensing value (S2(t)) in the first time stamp (t) and the second sensing value (S2(t+1)) in the second time stamp (t+1) consecutive to the first time stamp (t) is less than the reference value (D2).

The reason for comparing the second sensing value (S2(t+1)) in the second time stamp (t+1) with the reference value (D2) is that even if the user uses Heel Strike, the heel may not accidentally touch the floor (that is, the second sensing value (S2(t)) in the first time stamp (t) may be less than the reference value (D2)), and thus this is to clearly confirm that.

FIG. 12 is exemplary data for describing a gait analysis method according to some embodiments of the present invention. FIG. 12 is a case, in which the user uses Heel Strike.

Referring to FIG. 12, a time stamp column 801 is a first column, and is exemplarily displayed from 27 ms to 967 ms in 33 ms units.

In the left foot sensing value column 802, first sensing values (L_sensor1, L_sensor2, L_sensor3), and second sensing value (L_sensor4) at each time stamp are displayed. The first sensing values (L_sensor1, L_sensor2, and L_sensor3) may be a group of individual sensing values (L_sensor1, L_sensor2, and L_sensor3). Alternatively, unlike FIG. 12, the first sensing values (L_sensor1, L_sensor2, and L_sensor3) may be an individual sensing value (e.g., L_sensor1) selected from among individual sensing values (L_sensor1, L_sensor2, and L_sensor3). For example, FIG. 12 illustrates an example, in which the first sensor unit includes three sensors 201a, 202a, 203a (see FIG. 3), and FIG. 12 shows individual sensing values (L_sensor1, L_sensor2, L_sensor3) of each of the sensors 201a, 202a, and 203a.

In the left foot correction value column 803, correction values generated using the individual sensing values (L_sensor1, L_sensor2, and L_sensor3) of the first sensing values (L_sensor1, L_sensor2, and L_sensor3) at each time stamp are described. This correction value may be a product of individual sensing values (L_sensor1, L_sensor2, L_sensor3), a value generated based on the product, a sum or a value generated based on the sum, a weighted sum considering a weight, a weighted product, and the like. In FIG. 12, as an example, values generated based on the product of individual sensing values (L_sensor1, L_sensor2, and L_sensor3) are described.

In the right foot sensing value column 812, first sensing values (R_sensor1, R_sensor2, and R_sensor3) and second sensing value (R_sensor4) at each time stamp are displayed. The first sensing values (R_sensor1, R_sensor2, and R_sensor3) may be a group of individual sensing values (R_sensor1, R_sensor2, and R_sensor3). Alternatively, unlike FIG. 12, the first sensing values (R_sensor1, R_sensor2, and R_sensor3) may be an individual sensing value (e.g., R_sensor1) selected from among individual sensing values (R_sensor1, R_sensor2, and R_sensor3).

In the right foot correction value column 813, correction values generated using individual sensing values (R_sensor1, R_sensor2, and R_sensor3) of the first sensing values (R_sensor1, R_sensor2, and R_sensor3) at each time stamp are described. This correction value may be a product of individual sensing values (R_sensor1, R_sensor2, R_sensor3), a value generated based on the product, a sum or a value generated based on the sum, a weighted sum considering a weight, a weighted product, and the like. In FIG. 12, as an example, values generated based on the product of individual sensing values (R_sensor1, R_sensor2, and R_sensor3) are described.

In the right foot sensing value column 812, when comparing the second sensing value (R_sensor4) with a preset reference value (e.g., 50), the second sensing value (R_sensor4) at the time stamp of 357 ms is less than the preset reference value (i.e., 18<50) and the second sensing value (R_sensor4) at the time stamp of 390 ms is greater than a preset reference value (i.e., 100>50). Further, it can be seen that the first sensing values (R_sensor1, R_sensor2, and R_sensor3) at the time stamp of 390 ms are 0, and the second sensing values (R_sensor4) at the time stamp of 390 ms is 100. Thus, it can be seen that the user uses Heel Strike.

According to an embodiment of calculating the ground contact starting point, when comparing the second sensing value (R_sensor4) with a preset reference value (e.g., 50), the second sensing value (R_sensor4) at the time stamp of 357 ms is less than a preset reference value (i.e., 18<50), and the second sensing value (R_sensor4) at the time stamp of 390 ms is greater than a preset reference value (i.e., 100>50). Therefore, it can be predicted that the right foot lands from the time stamp of 390 ms instead of the time stamp of 357 ms. In other words, the ground contact starting point of the stance can be viewed as the time stamp of 390 ms.

According to another embodiment of calculating the ground contact starting point, a time between the time stamp 357 ms and the time stamp 390 ms may be predicted as the ground contact starting point of the stance. As described above, due to the limitation of the Bluetooth communication specification and the limitation of the power supply module (see 404 in FIG. 8) (i.e., using the primary/secondary battery), the first sensing values (R_sensor1, R_sensor2, R_sensor3), and second sensing value (R_sensor4) are provided for each time stamp. If the ground contact starting point is viewed as a point, at which the second sensing value (R_sensor4) is 50, a point of about 370 ms (between 357 ms and 390 ms) can be predicted as the ground contact starting point of the stance using interpolation. The interpolation may include linear interpolation, quadratic interpolation, Newton interpolation, Lagrange interpolation, and the like. When predicting the time between adjacent time stamps, various methods other than interpolation can be used. Using this method, it is possible to more accurately predict the ground contact starting point of the stance, beyond the limits of the Bluetooth communication specification/power supply module.

Referring to FIG. 12, when predicting the ground contact ending point of the stance, correction values of individual sensing values (R_sensor1, R_sensor2, R_sensor3) described in the right foot correction value column 813 may be used.

According to an embodiment, since the correction value described in the right foot correction value column 813 becomes 0 at the time stamp of 670 ms, it can be predicted that the right foot comes off from the ground from the time stamp of 670 ms. That is, the ground contact ending point of the stance can be viewed as the time stamp of 670 ms.

According to another embodiment, additionally, the correction value may be compared with a preset reference value (e.g., 3000). For example, a correction value at the time stamp of 637 ms is less than a preset reference value (i.e., 45<3000), and a correction value at the time stamp of 604 ms is greater than a preset reference value (i.e., 69225>3000). Therefore, it can be predicted that the right foot comes off from the ground from the time stamp of 604 ms. That is, the ground contact ending point of the stance can be viewed as the time stamp of 604 ms.

According to another embodiment, a time between the time stamp 604 ms and the time stamp 637 ms may be predicted as the ground contact ending point of the stance. If the ground contact ending point is viewed as the point, at which the correction value is 3000, the time point at about 632 ms (between 604 ms and 637 ms) can be predicted as the ground contact ending point of the stance using interpolation. Using this method, it is possible to more accurately predict the ground contact ending point of the stance, beyond the limits of the Bluetooth communication specification/power supply module.

Of course, when predicting the ground contact ending point of the stance, the individual sensing value (at least one of R_sensor1, R_sensor2, and R_sensor3) may be used without using the correction values of the individual sensing values (R_sensor1, R_sensor2, R_sensor3). For example, since the individual sensing value (e.g., R_sensor3) at the time stamp of 604 ms is 65 and the individual sensing value (e.g., R_sensor3) at the time stamp of 637 ms is 8, it can be seen the individual sensing value (e.g., R_sensor3) at the time stamp of 637 ms is less than the reference value (e.g., 50). Therefore, about 620 ms, which is a value between 604 ms and 637 ms, can be predicted as the ground contact ending point of the stance.

The ground contact time (GCT) can be obtained by subtracting the ground contact starting point from the ground contact ending point. For example, if the ground contact starting point (370 ms) is subtracted from the ground contact ending point (632 ms), the ground contact time (GCT) becomes 262 ms. By accurately predicting the ground contact ending point and the ground contact starting point, the ground contact time (GCT) can be accurately predicted.

Meanwhile, the stride length may be calculated using a track velocity of an electronic device (i.e., a tread mill). Specifically, the stride time between the ground contact ending point of the user's previous stance and the ground contact starting point of the user's current stance is calculated, and the user's stride length can be calculated using the track velocity and the stride time. Referring to FIG. 12, for example, if the ground contact ending point of the previous stance of the left foot is 230 ms, the ground contact starting point of the current stance of the left foot is 690 ms, and the track velocity is 10 km/h, the user's stride length is calculated as about 1.27 m (=(690 ms−230 ms)×10 km/h). Using data generated from different devices (i.e., shoes and a tread mill) as described above (i.e., the ground contact ending point of the previous stance generated from the shoe, the ground contact starting point of the user's current stance, and the track velocity generated from the tread mill), user's gait data can be accurately calculated.

FIG. 13 is exemplary data for describing a gait analysis method according to some embodiments of the present invention. FIG. 13 is a case, in which the user uses Forefoot Strike. For the convenience of description, descriptions with reference to FIG. 12 will be omitted.

Referring to FIG. 13, a time stamp column 801 is a first column, and is exemplarily displayed from 2871 ms to 3795 ms in units of 33 ms.

In the left foot sensing value column 802, first sensing values (L_sensor1, L_sensor2, L_sensor3), and second sensing value (L_sensor4) at each time stamp are displayed. In the left foot correction value column 803, correction values generated using the individual sensing values (L_sensor1, L_sensor2, and L_sensor3) of the first sensing values (L_sensor1, L_sensor2, and L_sensor3) at each time stamp are described. In the right foot sensing value column 812, first sensing values (R_sensor1, R_sensor2, and R_sensor3), and second sensing value (R_sensor4) at each time stamp are displayed. In the right foot correction value column 813, correction values generated using individual sensing values (R_sensor1, R_sensor2, and R_sensor3) of the first sensing values (R_sensor1, R_sensor2, and R_sensor3) at each time stamp are described.

In the right foot sensing value column 812, if any one individual sensing value (e.g., R_sensor1) of the first sensing values (R_sensor1, R_sensor2, and R_sensor3) is compared with a preset reference value (e.g., 50), the first sensing value (R_sensor1) at the time stamp 3267 ms is less than the preset reference value (i.e., 5<50), and the first sensing value (R_sensor1) at the time stamp of 3300 ms is greater than the preset reference value (i.e., 57>50). Further, at the time stamp of 3300 ms, since the first sensing value (R_sensor1) is greater than the second sensing value (R_sensor4), it can be seen that the user uses Forefoot Strike or Midfoot Strike (see step S724 in FIG. 11).

At the time stamps of 3300 ms and 3333 ms, since the second sensing value (R_sensor4) is less than a preset reference value (e.g., 50), it can be seen that the user uses Forefoot Strike instead of Midfoot Strike (see S726, S728 in FIG. 11).

According to an embodiment of calculating the ground contact starting point, if any one individual sensing value (e.g., R_sensor1) is compared with a preset reference value (e.g., 50), the individual sensing value (e.g., R_sensor1) at the time stamp of 3267 ms is less than a preset reference value (i.e., 5<50), and an individual sensing value (e.g., R_sensor1) at the time stamp of 3300 ms is greater than a preset reference value (i.e., 57>50). Therefore, it can be predicted that the right foot lands from the time stamp of 3300 ms instead of the time stamp of 3267 ms. In other words, the ground contact starting point of the stance can be viewed as the time stamp of 3300 ms.

According to another embodiment of calculating the ground contact starting point, a time between the time stamp 3267 ms and the time stamp 3300 ms may be predicted as the ground contact starting point of the stance. If the ground contact starting point is viewed as a point, at which any one individual sensing value (e.g., R_sensor1) is 50, the time point of about 3290 ms can be predicted as the ground contact starting point of the stance using interpolation. Using this method, it is possible to more accurately predict the ground contact starting point of the stance, beyond the limits of the Bluetooth communication specification/power supply module.

Referring to FIG. 13, when predicting the ground contact ending point of the stance, correction values of the individual sensing values (R_sensor1, R_sensor2, and R_sensor3) described in the right foot correction value column 813 are used.

According to an embodiment, since the correction value described in the right foot correction value column 813 is 0 at the time stamp of 3630 ms, it can be predicted that the right foot comes off from the ground from the time stamp 3630 ms. That is, the ground contact ending point of the stance can be viewed as the time stamp of 3630 ms.

According to another embodiment, additionally, the correction value may be compared with a preset reference value (e.g., 3000). For example, a correction value at the time stamp of 3597 ms is less than a preset reference value (i.e., 11<3000), and a correction value at the time stamp of 3564 ms is greater than a preset reference value (i.e., 3110>3000). Therefore, it can be predicted that the right foot comes off from the ground from the time stamp of 3597 ms. That is, the ground contact ending point of the stance can be viewed as the time stamp of 3597 ms.

According to another embodiment, a time between the time stamp of 3597 ms and the time stamp of 3564 ms may be predicted as the ground contact ending point of the stance. If the ground contact ending point is viewed as the point, at which the correction value is 3000, the time point of about 3569 ms can be predicted as the ground contact ending point of the stance using interpolation. Using this method, it is possible to more accurately predict the ground contact ending point of the stance, beyond the limits of the Bluetooth communication specification/power supply module.

Of course, when predicting the ground contact ending point of the stance, the individual sensing value (at least one of R_sensor1, R_sensor2, and R_sensor3) may be used without using the correction values of the individual sensing values (R_sensor1, R_sensor2, R_sensor3).

The ground contact time (GCT) can be obtained by subtracting the ground contact starting point from the ground contact ending point. For example, if the ground contact starting point (3290 ms) is subtracted from the ground contact ending point (3569 ms), the ground contact time (GCT) becomes 279 ms. By accurately predicting the ground contact ending point and the ground contact starting point, the ground contact time (GCT) can be accurately predicted.

As described above, the stride length may be calculated using a track velocity of an electronic device (i.e., a tread mill). Specifically, the stride time between the ground contact ending point of the user's previous stance and the ground contact starting point of the user's current stance is calculated, and the user's stride length can be calculated using the track velocity and the stride time.

FIG. 14 is an exemplary user interface displayed on an electronic device according to some embodiments of the present invention.

Referring to FIG. 14, an electronic device (for example, a tread mill) receives a first sensing value and a second sensing value sensed for each time stamp from the shoe 100, and determines the landing method of the user of the shoe based on the first sensing value and the second sensing value. If the determined landing method is the first landing method (for example, Heel Strike), the ground contact starting point of the stance is calculated by the first method (for example, calculated based on the second sensing value), and if the landing method is the second landing method (for example, Forefoot Strike or Midfoot Strike), the ground contact starting point of the stance is calculated by the second method (for example, calculated based on the first sensing value) different from the first method.

As shown in FIG. 14, on the user interface of the tread mill, exercise time, distance, velocity, pace, cadence, calories, and the like are displayed. In addition, the ground contact time (GCT) 820 of the left/right foot, the landing method 830, and the stride length 840 calculated in the manner described with reference to FIGS. 10 to 13 are displayed.

Further, the left and right GCT balances 825 may be displayed using the ratio of the ground contact time of the left foot/right foot. That is, if the ground contact time of the left foot/right foot is 150 and 150, respectively, the left and right GCT balance may represent 50, and if the ground contact time of the left and right foot is 120 and 180, the left and right GCT balance may represent 60 skewed to the right.

Meanwhile, the gait analysis method described with reference to FIGS. 10 to 14 may be stored in the form of a program (code) in a computer readable storage medium. Examples of the computer readable storage medium include ROM, RAM, CD-ROM, magnetic tape, floppy disk, optical data storage device, and the like, and also includes implementation in the form of a carrier wave (for example, transmission through the Internet). Further, the computer readable storage medium may be distributed over a computer system connected by a network, and computer readable codes may be stored and executed in a distributed manner.

Although the embodiments of the present invention have been described with reference to the above and the accompanying drawings, those of ordinary skill in the art to which the present invention pertains can understand that the present invention may be implemented in other specific forms without changing the technical spirit or essential features of the present invention. Therefore, it should be understood that the embodiments described above are illustrative and non-limiting in all respects.

What is claimed is:

1. A method for analyzing gait performed by an electronic device comprising:
receiving a first sensing value of a first sensor unit and a second sensing value of a second sensor unit sensed for each time stamp from a shoe including the first sensor unit corresponding to a ball of a foot or a toe and the second sensor unit corresponding to a heel;
determining a landing method of a user of the shoe based on the first sensing value and the second sensing value;
calculating a ground contact starting point of a stance by a first method if the determined landing method is a first landing method; and
calculating a ground contact starting point of a stance by a second method different from the first method if the determined landing method is a second landing method.

2. The gait analysis method of claim 1, wherein determining a landing method of a user of the shoe
based on the first sensing value and the second sensing value comprises,
performing, if any one of the first sensing value in a first time stamp and the second sensing value in the first time stamp is greater than a first reference value, at least one of comparing the first sensing value in the first time stamp and the second sensing value in the first time stamp, and comparing the second sensing value in the first time stamp and a second reference value, and
determining a landing method of a user of the shoe according to the comparison result.

3. The gait analysis method of claim 2, wherein determining a landing method of a user of the shoe according to the comparison result comprises,
determining that the user of the shoe uses Heel Strike if the second sensing value in the first time stamp is greater than the first sensing value in the first time stamp.

4. The gait analysis method of claim 2, wherein determining a landing method of a user of the shoe according to the comparison result comprises,
determining that the user of the shoe uses Midfoot Strike if the first sensing value in the first time stamp is greater than the second sensing value in the first time stamp, and the second sensing value in the first time stamp is greater than a second reference value.

5. The gait analysis method of claim 2, wherein determining a landing method of a user of the shoe according to the comparison result comprises,
determining that the user of the shoe uses Forefoot Strike if the first sensing value in the first time stamp is greater than the second sensing value in the first time stamp, and the second sensing value in a first time stamp and the second sensing value in a second time stamp consecutive to the first time stamp is less than a second reference value.

6. The gait analysis method of claim 2, wherein the first landing method is Heel Strike,
wherein a third time stamp is located immediately before the first time stamp,
wherein the first method determines a first time between the first time stamp and the third time stamp as a ground contact starting point of a stance by considering the second sensing value in the first time stamp and the second sensing value in the third time stamp.

7. The gait analysis method of claim 2, wherein the second landing method is Midfoot Strike or Forefoot Strike,
wherein a third time stamp is located immediately before the first time stamp,
wherein the second method determines a second time between the first time stamp and the third time stamp as a ground contact starting point of a stance by considering the first sensing value in the first time stamp and the first sensing value in the third time stamp.

8. The gait analysis method of claim 1, wherein a fifth time stamp is located immediately after a fourth time stamp, and the gait analysis method further comprises,
calculating a ground contact starting point of a stance; and
determining a third time between the fourth time stamp and the fifth time stamp as a ground contact ending point of a stance by considering the first sensing value in the fourth time stamp and the first sensing value in the fifth time stamp if the first sensing value in the fourth time stamp is greater than a third reference value and the first sensing value in the fifth time stamp is less than a third reference value.

9. The gait analysis method of claim 1, wherein the first sensor unit includes at least two sensors, and the first sensing value is a collection of individual sensing values sensed by each of the at least two sensors,
wherein the fifth time stamp is located immediately after the fourth time stamp, and the gait analysis method further comprises,
calculating a ground contact starting point of a stance,
determining a third time between the fourth time stamp and the fifth time stamp as a ground contact ending point of a stance by considering correction values of individual sensing values in the fourth time stamp and correction values of individual sensing values in the fifth time stamp if a correction value generated using the individual sensing values in the fourth time stamp is greater than a fourth reference value, and a correction value generated using the individual sensing values in the fifth time stamp is less than a fourth reference value.

10. The gait analysis method of claim 1, wherein the electronic device is a tread mill, and the gait analysis method further comprises,
receiving a track velocity of the tread mill,
calculating a stride time between a ground contact ending point of a user's previous stance and a ground contact starting point of a user's current stance, and
calculating a stride length of the user using the track velocity and the stride time.

11. The method for claim 1, wherein the first landing method is Heel Strike, and the first method is to calculate a ground contact starting point of the stance based on a second sensing value,
wherein the second landing method is Forefoot Strike or Midfoot Strike, and the second method is to calculate a ground contact starting point of the stance based on the first sensing value.

12. A computer readable storage medium storing instructions of a computer program for implementing the gait analysis method of claim 1.

13. An electronic device comprising:
a communication module;
a processor; and
a memory,
wherein the memory stores instructions causing the communication module to receive a first sensing value of a first sensor unit and a second sensing value of a second sensor unit sensed for each time stamp from a shoe including the first sensor unit corresponding to a ball of a foot or a toe and the second sensor unit corresponding to a heel,
wherein the memory stores instructions causing the processor to,
determine a landing method of a user of the shoe based on the first sensing value and the second sensing value,
calculate a ground contact starting point of a stance by a first method if the determined landing method is a first landing method, and
calculate a ground contact starting point of a stance by a second method different from the first method if the determined landing method is a second landing method.

14. The electronic device of claim 13, wherein the electronic device is a tread mill,
wherein the memory further stores instructions causing the processor to,
receive a track velocity of the tread mill,
calculate a stride time between a ground contact ending point of a user's previous stance and a ground contact starting point of a user's current stance, and
calculate a user's stride length using the track velocity and the stride time.
